(19) United States
(12) Patent Application Publication
(10) Pub. No.: US 2022/0176006 A1
(43) Pub. Date: Jun. 9, 2022

(54) WALL-MOUNTED PHOTOCATALYTIC-OXIDIZING AIR PURIFIER

(71) Applicant: Wynd Technologies, Inc., San Jose, CA (US)

(72) Inventors: Jason Duy Vuu, San Jose, CA (US); Eric Munoz, South San Francisco, CA (US); Raymond Rui Wu, Millbrae, CA (US)

(21) Appl. No.: 17/540,523

(22) Filed: Dec. 2, 2021

Related U.S. Application Data
(60) Provisional application No. 63/120,894, filed on Dec. 3, 2020.

(51) Int. Cl. A61L 9/20

(52) U.S. Cl. CPC A61L 9/205; A61L 2209/12; A61L 2209/15

(57) ABSTRACT

Air purification systems and methods use a flat, wall-mounted enclosure containing a photocatalytic oxidation (PCO) system to remove airborne contaminants. The horizontal width and vertical height of the PCO system being large compared to its thickness to maximize catalytic surface area and efficiently use light to drive reactions. PCO performance may be further optimized through contouring a catalytic panel to increase surface area. The structure and does not require floor or counter space, and a visible face of the wall-mounted structure may have an aesthetic feature.

### REFERENCE TO RELATED APPLICATIONS

This patent document claims benefit of the earlier filing date of U.S. Provisional Pat. App. No. 63/120,894, entitled “Photocatalytic Oxidation System for Removal of Indoor Pollutants within Wall-Mounted Decorative Structures,” filed Dec. 3, 2020, which is hereby incorporated by reference in its entirety.

### BACKGROUND OF THE INVENTION

Photocatalytic oxidation (PCO) is a known technique for removing volatile organic compounds (VOCs) such as formaldehyde, methane, chlorides, and benzene from air. PCO generally involves exposing a catalytic surface such as a titanium dioxide (TiO₂) surface to ultraviolet (UV) light. In general, the UV light breaks chemical bonds in compounds that are in contact with the catalytic surface. For example, UV light interacts with water vapor (H₂O) at a TiO₂ surface to produce reactive OH⁻ ions that can oxidize VOCs. An efficient PCO process can convert the VOCs into more basic and non-toxic compounds such as carbon dioxide (CO₂) and water.

Current PCO-based air purifiers have limitations. PCO-based air purifiers particularly need a controlled air flow. If air flow moves too quickly, the desired reactions do not occur or are incomplete, and the purifier could even create and release additional toxic pollutants. Many current PCO-based air purifiers also have mesh-shaped catalyst that allow air to pass through large gaps without interacting with the catalyst, and as a result, the air purifiers may not provide enough catalytic surface area that interacts to provide effective one-pass air cleaning. Another problem is that current PCO air purifiers are mostly standing devices that take up floor or counter space in homes or establishments using the PCO air purifiers.

### SUMMARY OF THE INVENTION

In accordance with an aspect of the present disclosure, a PCO system has a thin, flat structure that may be wall mounted and disguised with a decorative face or feature such as a painting or other aesthetically pleasing wall decoration. The wall mounting saves space, and the flat geometry of the PCO efficiently produces oxidation reactions with a large continuous surface area catalyst. The PCO system may include a distributed or large-area UV-light source positioned to directly irradiate a catalytic sheet, e.g., a titanium dioxide (TiO₂) sheet or TiO₂-coating on a sheet of another material. The catalytic sheet may have surface variations such as undulations, projections, or villi-like structures to further increase catalytic surface area for improved reactions. The flat PCO structure may also provide wide air inlets and outlets for an effective air flow even at slow air velocities. Ambient air passes in between the UV lighting sheet and the catalytic sheet, and heating of air in the PCO system, which is inherent to the catalyzed reactions and absorption of UV light, drives or helps move air upward through the PCO system. In some configurations, uneven heating, e.g., a non-uniform UV light intensity emission from the UV sheet, may create a temperature gradient that improves air flow.

### BRIEF DESCRIPTION OF THE DRAWINGS

FIG. 1 is a cross-sectional view of a wall-mounted photocatalytic oxidation (PCO) air purifier in accordance with an example of the present disclosure.

FIG. 2 shows a distributed UV light source for a PCO air purifier in accordance with an example of the present disclosure.

FIG. 3 illustrates surface variations in a catalyst for a PCO air purifier in accordance with an example of the present disclosure.

FIG. 4 shows a decorative face of a wall-mounted PCO system in accordance with example of the present disclosure,

The drawings illustrate examples for the purpose of explanation and are not of the invention itself. Use of the same reference symbols in different figures indicates similar or identical items.

### DETAILED DESCRIPTION

Highly efficient air purification systems and methods use a flat, wall-mounted enclosure containing a photocatalytic oxidation (PCO) system to remove airborne contaminants such as volatile organic compounds (VOCs) from the air. The horizontal width and vertical height of the PCO system being large compared to its thickness of the PCO system maximizes area available for a catalyst of light-driven reactions. PCO performance may be optimized through increasing the surface area of the catalyst and reducing the air flow speed, which results in efficient pollutant contact with the catalytic surface where UV light is incident. The structure and does not require floor or counter space for PCO air purification, and a visible face of the wall-mounted structure may have an integrated or user-selected aesthetic feature to make the air purification system a desirable addition to a room or other interior environment.

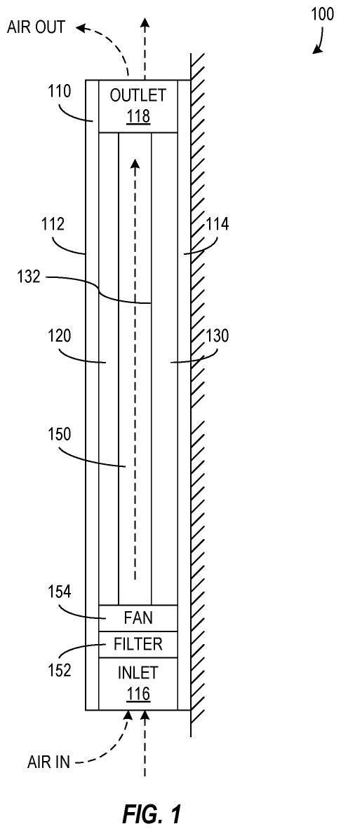

FIG. 1 shows a cross-sectional view of a PCO-based air purifier 100 in accordance with an example of the present disclosure. Air purifier 100 has a flat enclosure including a front face 112, a wall mounting structure 114, an air inlet 116, and an air outlet 118. As describe further below front face 112 may include decorative or aesthetic features to enhance the look of air purifier 100 when mounted on a wall. Wall mounting structure 114 is on or forms a back face of enclosure 110 and may include any conventional wall mount that is able to attach air purifier 100 to a wall, e.g., to an interior wall of a home, an office, or other structure, and is able to bear the weight of air purifier 100. Wall mounting structure 114 may, for example, include screws, lag bolts, nails, wall anchors, or hooks and wire, or picture hangings systems that are able to secure air purifier 100 on a wall.

Enclosure 110 and air purifier 100 are generally flat having vertical and horizontal dimensions up to about 20 cm to 2 m or more and thickness about 1 to 10 cm or less. More generally, while air purifier may be box shaped with horizontal and vertical sides forming edges of a square or a rectangle, air purifier 100 may have any shape, e.g., square, rectangular, circular, oval, polygonal, star-shaped, or irregularly shaped. Further, the size and shape of air purifier 100 may be chosen according to the desired air purification capabilities for air purifier 100, the available wall space where air purifier 100 will be install, or aesthetic inclinations. Air inlet 116 and air outlet 118 are generally narrow to provide a thin air purifier profile but may run substantially the entire horizontal width of enclosure 110 to maximize air throughput at low air velocity.

PCO air purifier 100 internally includes a UV light panel 120 and a catalytic panel 130 that are separated by an air channel or gap 150. Air channel 150 is thin e.g., less than about 1 cm, but has an area that is substantially the same as the wall area of air purifier 100. In operation, UV light panel 120 directs UV light in a roughly horizontal direction through air channel 150 onto a surface 132 of catalytic panel 130 while ambient air flows vertically upward through channel 150. More specifically, ambient air from the room containing air purifier 100 enters air channels 150 via inlet 116 at the bottom of air flow channels 150, flows upward along surface 132 of catalytic panel 130 while being irradiated with UV light from UV light panel 120, and exits through outlet 118 at the top of air channel 150. Optionally, inlet system 116 or outlet system 118 may have associated air filter and movement systems. For example, a filter system 152 may include a high-efficiency particulate air (HEPA) filter that removes particulates from the ambient air passing through air purifier 100. An air filter system 152 may remove particulates, while photocatalytic reactions remove chemicals such as VOCs that may pass through filter system 152. Alternatively, the photocatalytic process may oxidize particulates, but filter system 152 may advantageously prevent particles from entering channel 150 to avoid or reduce fouling of light panel 120 or catalytic panel 130. An air movement system 154 may include an array of small diameter fans lined up along the length of inlet 116 or one or more squirrel cage fans or blowers. In general, whether air movement system 154 is needed in air purifier 100 may depend on the air flow that convection provides, the maximum air velocity providing efficient photocatalytic reactions, and the minimum air velocity needed to achieve a desired air purification rate.

UV light panel 120 includes one or more light sources that produce UV light that is directed at catalytic panel 130 and distributed across the area of catalytic panel 130. UV light panel 130 may for example include an array of light emitting diodes (LEDs) or laser diodes that emit that collectively emit one or more of UV-A light having wavelengths between about 320 nm and about 400 nm, UV-B light having wavelengths between about 290 nm and about 320 nm, or UV-C light having wavelengths between about 100 nm and about 290 nm. For example, UV-A and UV-C wavelengths may be used for TiO₂ as the catalyst in catalytic panel 130. UV-A light is generally safer if humans may be exposed to UV light that escapes from air purifier 100, but UV-C light may be safely confined inside air purifier 100 and used to kill or deactivate airborne germs or virus that enter air purifier 100.

The intensity of light from UV light panel 120 is related to the optimal air velocity through air purifier 100. In general, the airflow may intentionally be kept slow to allow for the complete decomposition of VOCs. An air flow rate may be about 1 CFM for every 10 W/m2 incident on catalytic panel 130.

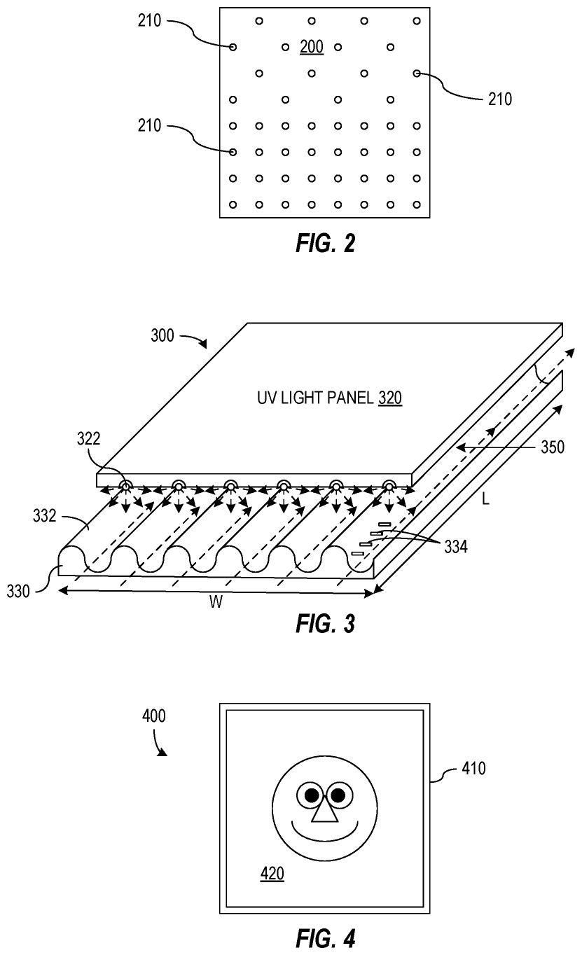

Energy from UV light panel 120 and from the catalyzed reactions may heat air inside air channel 150 resulting in convection where the heated air rises up and out of outlet 118 and draws ambient air in through inlet 116 at the bottom of air purifier 100. The heating may be achieved using a nominally uniform spatial distribution of the light from UV light panel 120. In accordance with an aspect of the current disclosure, a UV light panel that produces UV light with a non-uniform, spatial intensity distributions may drive convective airflow in an air purifier. FIG. 2 shows an example of a UV light panel 200 having UV light sources 210 that are asymmetrically distributed across the area of panel 200 to produce a non-uniform spatial intensity distribution. UV light sources 210 may, for example, be UV LEDs having substantially the same power output, but UV light sources 210 are more densely packed at the bottom of UV light panel 200. The non-uniform distribution of UV light from panel 200 may tend to heat the bottom of the catalytic panel more and create a positive temperature gradient (hotter at the bottom than the top), and the temperature gradient may help drive an airflow from bottom-up in an air purifier. A fan or other air movement system can be added to an air purifier if the air purifier requires more air flow convection alone provides.

Air flows inside air purifier 100 of FIG. 1 through one or more channels 150 between UV light panel 120 and catalytic panel 130, and surface 132 of catalytic panel 130, while the air and catalytic sheet 130 is exposed to the UV light. A PCO reaction occur where air, e.g., VOC pollutants, oxygen, and water vapor in the air, contacts surface 132. This PCO reaction changes VOCs in the air into benign chemical compounds that exit air purifier 100 with the natural flow of air out of outlet 118 at the top of air purifier 100. UV light in air purifier 100 of FIG. 1 needs to be incident on the entire surface 132 of catalytic panel 130 for full utilization of catalytic panel 130. For a light panel such as UV light panel 120 including multiple discrete light sources 210, one or more optical systems may be coupled to light sources 210 to spread UV light across surface 132 of catalytic panel 130 and increases the reaction rate by increasing the surface area where reactions occur. Additionally, increasing the area of surface 132 that is exposed to UV light can increase the reaction rate.

In accordance with an aspect of the current disclosure, surface 132 of catalytic panel 130 includes a continuous sheet that may be contoured to increase catalytic surface area that enclosure 110 of air purifier 100 can accommodate. Surface 132 may, for example, be pleated or textured to increase surface area and maximize the efficiency of the PCO process. A catalytic panel may be layered or textured to increase the area available for catalyzed reactions. A layered catalytic panel may have one or more layers that are exposed to air flow and semitransparent to pass UV to underlying layers. Additionally, a catalytic panel may include a reflective surface to reflect some UV light back through translucent or transmissive layers or reflect some UV light to catalytic surface areas that might not receive direct UV light from the light panel. FIG. 3 shows an example of a PCO system 300 including a UV light panel 320 and a catalytic panel 330, where catalyst 330 has a contoured surface 310 that is wave shaped to increase the catalytic surface area that may be provided within a horizontal width W of an air channel 350 between catalytic panel 330 and UV light panel 320. Catalytic panel 330 may also be contoured with waves along a vertical length L of air channel 350 between catalytic panel 330 and UV light panel 320. The combination of wave-shaped contours in horizontal and vertical directions provides a continuous catalytic surface 332 with an array of dimples or hills. With or without contouring along the vertical direction, the wave shape of catalytic panel 330 ensures airflow has one or more pathways along catalytic surface 332 that air must traverse to complete passage through PCO system 300.

PCO system 300 illustrates only one example of contoured surface shaped to increase the catalytic surface area available within a specific horizontal width W and vertical length L. Some additional or alternative ways to provide increased catalytic surface area include any repetitive shape contours, e.g., a saw shape. Additionally, a catalytic surface may include fingers or villi-like projections, e.g., TiO₂ rods or threads, extending from an otherwise continuous flat or contoured surface. Projections 334 on wave-shaped catalytic surface 332, for example, may increase the reactive surface area of catalytic panel 330. Fractal surfaces may be most efficient to maximize the reactive surface area of a catalytic sheet contained within a specific width and length.

FIG. 3 also illustrates how one or more light sources 322 on a UV light panel 320 may be arranged or may have associated optical systems that provide light tailored to the shape of catalytic panel 330. In particular, light from UV panel may have a spatial and directional distribution such that most light incident on catalytic panel 330 is perpendicular to surface 332 or such that the incident light intensity is approximately uniform at least along the horizontal direction on surface 332.

Air purifiers such as air purifier 100 of FIG. 1 may be thin and wall-mounted and therefore may be unobtrusive in a room where the air purifier is installed. An air purifier may further have one or more features to disguise or enhance the appearance of the purifier once the air purifier is installed in a home or office. For air purifier 100, enclosure 110 and particularly front face 112 may include a decorative feature. For example, enclosure 110 may include a picture frame extending along the sides, top, and bottom of enclosure 110, and the picture frame around front face 112 may hold a painting, photograph, or other artistically pleasing image that air purifier 100 displays in the room or office where air purifier 100 is installed. The displayed image may be factory installed with one option or multiple options from which a user may select. Alternatively, an image may be changeable or removable to allow a user to switch images throughout the lifetime of air purifier 100. Instead of using a simple image, the aesthetic feature on front face 112 may hold a sculpture or relief. Front face 112 may further include an aesthetic feature that also provides utility. For example, front face 112 may include a mirror, a clock face, or a shelf on which useful or decorative items may be displayed.

FIG. 4 shows a front view of an air purifier 400 having an enclosure with sides, top, and bottom forming a frame 410 that holds an aesthetic feature 420, e.g., a drawing, a painting, a poster, a print, or a photograph.

Although particular implementations have been disclosed, these implementations are only examples and should not be taken as limitations. Various adaptations and combinations of features of the implementations disclosed are within the scope of the following claims.

What is claimed is:

1. A air purifier comprising:
an enclosure including a wall mounting structure on a back face of the enclosure, an air inlet on a bottom of the enclosure, and an air outlet on a top of the enclosure;
a catalytic panel inside the enclosure and having a catalytic surface;
a light panel inside the enclosure and defining, between the light panel and the catalytic panel, an air channel that directs air flow from the air inlet along the catalytic surface to the air outlet, the light panel directing light through the air channel onto the catalytic surface to induce photocatalytic reactions.

2. The air purifier of claim 1, wherein a first distance between the back face and a front face of the enclosure is less than a second distance between the air inlet and the air outlet.

3. The air purifier of claim 2, wherein a third distance between vertical sides of the enclosure is greater than the first distance.

4. The air purifier of claim 2, wherein the first distance is less than 10 cm, and the second distance and the third distance are more than 20 cm.

5. The air purifier of claim 1, wherein the catalytic surface has a contour that increases a total area of the catalytic surface receiving light from the light panel.

6. The air purifier of claim 5, wherein the contour is a wave shape.

7. The air purifier of claim 5, wherein the catalytic surface includes a plurality of projections projecting from the contour.

8. The air purifier of claim 7, wherein the projections comprise rods or threads.

9. The air purifier of claim 1, wherein the light panel directs UV light onto the catalytic surface.

10. The air purifier of claim 1, further comprising an aesthetic feature on a font face of the enclosure.

11. The air purifier of claim 10, wherein the aesthetic feature comprises at least one of a painting, a drawing, a print, and a photograph.

12. The air purifier of claim 1, wherein the enclosure further comprises a frame holding a removable image on a front face of the enclosure.
